(19) United States
(12) Patent Application Publication
(10) Pub. No.: US 2023/0383364 A1
(43) Pub. Date: Nov. 30, 2023

(54) PROGNOSTIC MODEL OF HEPATOCELLULAR CARCINOMA BASED ON DDR AND ICD GENE EXPRESSION AND CONSTRUCTION METHOD AND APPLICATION THEREOF

(71) Applicant: The First Affiliated Hospital of Sun Yat-Sen University, Guangzhou (CN)

(72) Inventors: Jiaping Li, Guangzhou (CN); Xiaokai Zhang, Guangzhou (CN); Jie Wen, Guangzhou (CN)

(21) Appl. No.: 18/310,938

(22) Filed: May 2, 2023

(30) Foreign Application Priority Data
May 5, 2022 (CN) 202210481679.5

(51) Int. Cl. C12Q 1/6886; G16B 40/00; G16H 50/20; G16B 5/20; G16B 20/00

(52) U.S. Cl. CPC C12Q 1/6886; G16B 40/00; G16H 50/20; C12Q 2600/118; G16B 20/00; C12Q 2600/158; G16B 5/20

(57) ABSTRACT

The present disclosure relates to a construction method for a prognostic model of hepatocellular carcinoma based on DNA damage repair (DDR) and immunogenic cell death (ICD) gene expression, including the following steps of: Step 1, acquiring transcription profile expression data of multiple hepatocellular carcinoma patients; step 2, screening candidate genes based on the transcription profile expression data of multiple hepatocellular carcinoma patients; step 3, determining prognostic genes related to lifetime through single-factor Cox regression analysis based on the candidate genes; step 4, screening the genes related to the lifetime through LASSO Cox regression analysis; and step 5, assessing the prediction performance of the risk score model based on the above training dataset.

### TECHNICAL FIELD

The present disclosure relates to the technical field of hepatocellular carcinoma tumors, particular to a prognostic model of hepatocellular carcinoma based on DNA damage repair (DDR) and immunogenic cell death (ICD) gene expression and a construction method and an application thereof.

### BACKGROUND

The liver is one of the important organs maintaining in vivo environmental stability and body health. Hepatocellular carcinoma is the most common malignant tumor of the liver and one of the leading causes of death. Every year, almost one million people die as a result of liver cirrhosis and hepatocellular carcinoma. It should be noted that as the most common primary malignant tumor in the liver, hepatocellular carcinoma has been at the forefront of various diseases that cause human death due to high morbidity, difficulty diagnosis, and limited treatment options. Hepatocellular carcinoma currently ranks fifth in terms of tumor mortality worldwide, and it is the leading cause of death in some African and Asian countries.

Hepatocellular carcinoma research has advanced significantly in recent years. In the previous century, “early treatment of small hepatocellular carcinoma” and “two-stage resection of hepatocellular carcinoma after shrinking” each contributed 10 percentage points to the improvement of the hepatocellular carcinoma postoperative survival rate. However, because of its rapid progression and extremely high recurrence rate, hepatocellular carcinoma has a poor overall treatment efficacy, and the hepatocellular carcinoma patient population has only about 5% of the overall 5-year survival rate. Despite some progress in basic and clinical research of hepatocellular carcinoma in recent years, a recurrence mechanism of hepatocellular carcinoma has not been clarified, and effective intervention measures have not been discovered. The high recurrence rate of hepatocellular carcinoma has become the bottleneck in improving its therapeutic effect. As a result, the search for biomarkers associated with the prognostic recurrence of hepatocellular carcinoma may provide a new method for further lowering the recurrence rate and mortality of clinical hepatocellular carcinoma.

Hepatocellular carcinoma treatment methods primarily include surgical resection, local treatment such as ablation and chemoembolization, and systemic treatment such as chemotherapy, targeting, and immunotherapy. Whereas chemotherapy plays an important role in the treatment of middle and advanced hepatocellular carcinoma, patients have different treatment responses to chemotherapy, resulting in a significant difference in the prognosis of patients receiving the same treatment. Currently, clinicians predict the prognosis of hepatocellular carcinoma patients and guide clinical treatment plans based on patients' clinical manifestations, liver function reserves, and tumor markers determination, but their differences are large, which frequently leads to a deviation between clinical judgment and actual situations, affecting patient treatment or resulting in ineffective treatment. As a result of the ongoing advancement and popularization of gene detection technology, it is now possible to predict the prognosis of hepatocellular carcinoma patients based on level of gene expression, resulting in more accurate clinical judgment. We developed a prediction model for the prognosis of hepatocellular carcinoma combining gene detection data and prognosis data from hepatocellular carcinoma patients in the public database, which has potential application prospects in the classification treatment and prognostic prediction of hepatocellular carcinoma.

The Chinese patent CN113,345,589A provides a method for developing a hepatocellular carcinoma prognostic model as well as its application method and electronic equipment. The method includes: collecting transcription profile expression data from multiple hepatocellular carcinoma patients and multiple reference individuals; screening candidate genes based on the transcription profile expression data; and building a risk score model based on the candidate genes. A risk score model is included in the prognostic models of hepatocellular carcinoma.

This method uses transcription profile expression data to screen the candidate genes for construction models. This method is inadequate in constructing models of DDR genes and ICD-related genes, and the treatment and prognosis effects are poor and need to be further improved.

To that end, the present disclosure provides a hepatocellular carcinoma prognostic model based on DDR and ICD gene expression as well as a construction method and an application thereof.

### SUMMARY

Based on the defects in the prior art, the present disclosure aims at providing a prognostic model of hepatocellular carcinoma based on DDR and ICD gene expression, as well as a construction method and application thereof, to solve the problems proposed in the background art.

To address the technical problems raised by the present disclosure, the following technical solution is used:

The present disclosure provides a construction method for a prognostic model of hepatocellular carcinoma based on DNA and (ICD) gene expression, including the following steps:

step 1, obtaining transcription profile expression data from multiple hepatocellular carcinoma patients;

step 2, screening candidate genes based on the transcription profile expression data of the hepatocellular carcinoma patients;

step 3, determining prognostic genes related to lifetime through single-factor Cox regression analysis based on the candidate genes;

step 4, screening the genes related to the lifetime through LASSO Cox regression analysis to determine genes for constructing a risk score model and the risk score model; and

step 5, assessing the prediction performance of the risk score model based on the above training dataset.

Preferably, the genes for constructing the risk score model comprise FFAR3, DDX1, POLR3G, FANCL, ADA, PIK3R1, DHX58, TPT1, MGMT, SLAMF6, and EIF2AK4.

Preferably, step 5 comprises:

calculating a risk score of each subject in the training dataset based on the risk score model;

analyzing the score by using a time-dependent subject working characteristic curve of the training dataset; and

analyzing and evaluating the fitting goodness of the score model by using the time-dependent subject working characteristic curve of the training dataset.

Preferably, a grouping cut-off value is analyzed and determined according to the time-dependent subject working characteristic curve of the training dataset, and the subjects in the training dataset are divided into a first high-risk group and a first low-risk group according to the grouping cut-off value;

whether the first high-risk group and the first low-risk group have a significant difference in survival is assessed using the Kaplan-Meier curve of the training dataset.

Preferably, cox regression analysis includes single-factor cox analysis and multi-factor cox analysis.

Preferably, the single-factor cox analysis is as follows:

regression modeling is respectively performed on a single gene or a clinical characteristic by using a coxph function of a survival package to screen prognosis-related genes or clinical characteristics based on p<0.01, corresponding modeling parameters are extracted and then a forest map is plotted by using a forest plot package.

the multi-factor cox analysis is as follows: regression modeling is performed on the constructed multi-gene or clinical characteristics by using a coxph function of a survival package.

Preferably, in the LASSO Cox regression,

LASSO regression modeling is performed on prognosis-related genes by using a glmnet function of R package glmnet, and cross-validation is performed on a cv.glmnet function;

LASSO screening is performed by using lambda.min as an optimal lambda parameter to obtain 21 genes, wherein a multivariable cox model is screened further stepwise, and 11 genes are finally retained, the multi-factor cox model is constructed using these genes, and the corresponding risk score is calculated.

Preferably, the independent validation and nomogram of the risk score are as follows:

first, single-factor cox analysis is performed on the TCGA-LIHC dataset in combination with clinical pathological characteristics: stage, gender, vascular, age, and AFP;

second, multi-factor cox regression is utilized to analyze the above 6 factors including the overall prognosis of risk score to validate the independent prognosis effect of risk score;

a cox proportional risk regression model is constructed by using a cph function of R package rms, then survival probability is calculated by using a survival package, a nomogram is finally constructed by using a nomogram function, and a calibration curve is plotted to assess the nomogram and predict accuracy.

The present disclosure also includes a hepatocellular carcinoma prognostic model obtained by employing a hepatocellular carcinoma prognostic model construction method based on gene expression.

The present disclosure also includes an application of a method for building a hepatocellular carcinoma prognostic model based on gene expression in the treatment and prognosis of hepatocellular carcinoma.

In comparison to the prior art, the present disclosure has the following advantages:

The hepatocellular carcinoma prognostic model provided by the present disclosure is built on DDR and ICD-related genes, which are based on first-line drugs for clinical hepatocellular carcinoma chemotherapy. These DNA damage drugs can lead to a wide range of DDR and ICD effects on hepatocellular carcinoma cells, and affecting treatment response and prognosis.

### BRIEF DESCRIPTION OF THE DRAWINGS

FIG. 1 is an expression heat map of ICD&DDR-related genes in TCGA-LIHC of the present disclosure.

FIGS. 2(a)-2(c) shows LASSO regression modeling and parameter adjustment optimization of prognosis-related ICD&DDR genes of the present disclosure.

FIG. 3(a) is a nomogram of the present disclosure, FIG. 3(b) and FIG.3(C) are 1-year and 3-year correction curves of the present disclosure.

The patent or application file contains at least one drawing executed in color. Copies of this patent or patent application publication with color drawing(s) will be provided by the Office upon request and payment of the necessary fee.

### DETAILED DESCRIPTION OF THE EMBODIMENTS

Next, in the embodiments of the present disclosure the technical solution will be clearly and completely described in conjunction with accompanying drawings. The described embodiments are only some of the embodiments of the present disclosure, not all of them. Other embodiments obtained by persons of ordinary skill in the art without creative efforts are all included within the scope of protection of the present disclosure based on the embodiments of the present disclosure.

### EXAMPLE 1

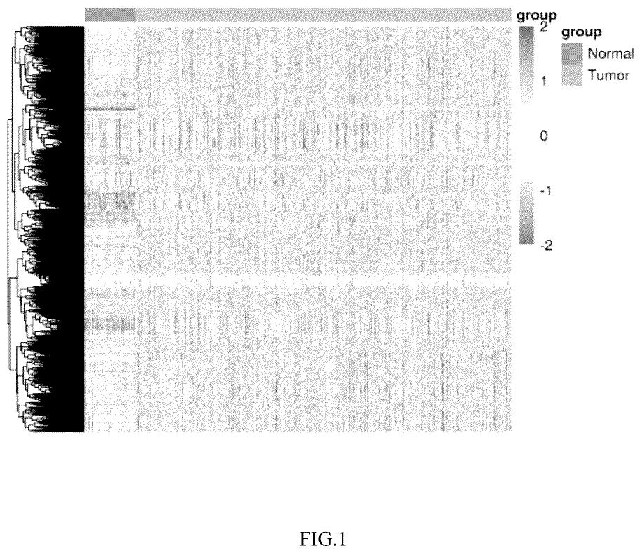

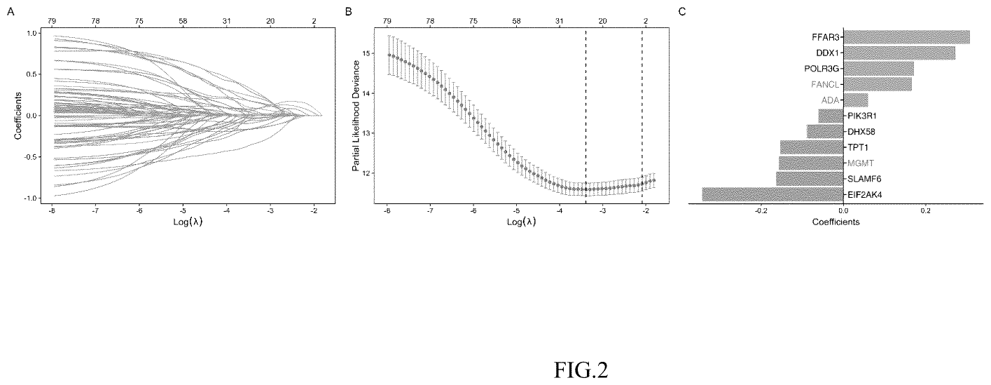

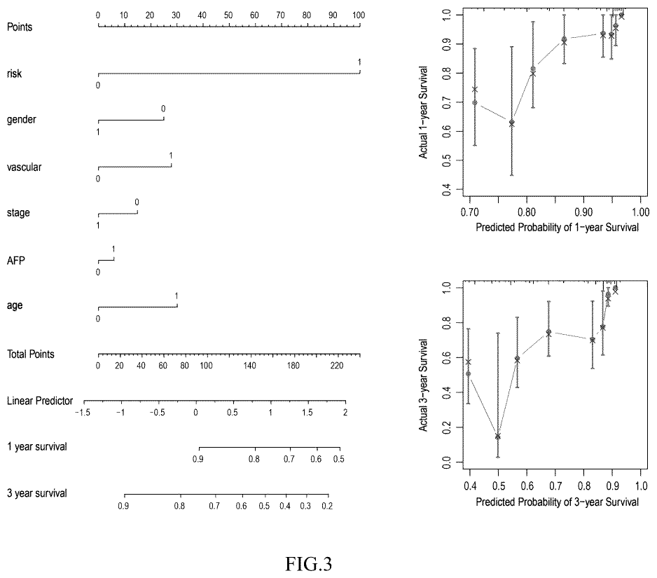

Referring to FIG. 1-FIG. 3, this example provides a construction method of a prognostic model of hepatocellular carcinoma based on gene expression, the construction method comprising the following steps:

step 1, obtaining transcription profile expression data from multiple hepatocellular carcinoma patients;

step 2, screening candidate genes based on the transcription profile expression data of the multiple hepatocellular carcinoma patients;

step 3, determining prognostic genes related to lifetime through single-factor Cox regression analysis based on the candidate genes;

step 4, screening the genes related to the lifetime through LASSO Cox regression analysis to determine genes for constructing a risk score model and the risk score model; and

step 5, assessing the prediction performance of the risk score model based on the above training dataset.

In this example, the genes for constructing the risk score model comprise FFAR3, DDX1, POLR3G, FANCL, ADA, PIK3R1, DHX58, TPT1, MGMT, SLAMF6, and EIF2AK4.

In this example, step 5 includes:

calculating a risk score of each subject in the training dataset based on the risk score model;

analyzing the score by using a time-dependent subject working characteristic curve of the training dataset; and

analyzing and evaluating the fitting goodness of the score model by using the time-dependent subject working characteristic curve of the training dataset.

In this example, a grouping cut-off value is analyzed and determined according to the time-dependent subject working characteristic curve of the training dataset, and the subjects in the training dataset are divided into a first high-risk group and a first low-risk group according to the grouping cut off value.

whether the first high-risk group and the first low-risk group are of significant difference in the aspect of survival is assessed by using the Kaplan-Meier curve of the training dataset.

In this example, the Cox regression analysis comprises single-factor cox analysis and multi-factor cox analysis.

In this example, the single-factor cox analysis is as follows:

regression modeling is respectively performed on a single gene or a clinical characteristic by using a coxph function of a survival package to screen prognosis-related genes or clinical characteristics based on p<0.01, corresponding modeling parameters are extracted and then a forest map is plotted by using a forest plot package;

the multi-factor cox analysis is as follows: regression modeling is performed on the constructed multi-gene or clinical characteristics by using a coxph function of a survival package.

In this example, in the LASSO Cox regression,

LASSO regression modeling is performed on prognosis-related genes by using a glmnet function of R package glmnet, and cross-validation is performed on a cv.glmnet function;

LASSO screening is performed by using lambda.min as an optimal lambda parameter to obtain 21 genes, wherein a multivariable cox model is screened further stepwise, and 11 genes are finally retained, the multi-factor cox model is constructed using these genes, and the corresponding risk score is calculated.

In this example, the independent validation and nomogram of the risk score is as follows:

first, single-factor cox analysis is performed on the TCGA-LIHC dataset in combination with clinical pathological characteristics: stage, gender, vascular, age, and AFP;

second, multi-factor cox regression is utilized to analyze the above 6 factors including the overall prognosis of risk score to validate the independent prognosis effect of risk score;

a cox proportional risk regression model is constructed by using a cph function of R package rms, then survival probability is calculated by using the survival package, a nomogram is finally constructed by using a nomogram function, and a calibration curve is plotted to assess the nomogram and predict accuracy.

This example provides a hepatocellular carcinoma prognostic model obtained by employing a hepatocellular carcinoma construction method based on DDR and ICD gene expression.

This example provides an application of a hepatocellular carcinoma prognostic model construction method based on DDR and ICD gene expression in the treatment and prognosis of hepatocellular carcinoma.

### EXAMPLE 2

This example provides a hepatocellular carcinoma prognostic model construction method based on gene expression, the construction method includes the following steps:

step 1, obtaining transcription profile expression data of multiple hepatocellular carcinoma patients;

step 2, screening candidate genes based on the transcription profile expression data of multiple hepatocellular carcinoma patients;

step 3, determining prognostic genes related to lifetime through single-factor Cox regression analysis based on the candidate genes;

step 4, screening the genes related to the lifetime through LASSO Cox regression analysis to determine genes for constructing a risk score model and the risk score model; and

step 5, assessing the prediction performance of the risk score model based on the above training dataset.

In this example, TCGA-LIHC, GSE14520, and ICGC LIRI-JP data, variation data, clinical information, and follow-up visit information, and the like are downloaded from XENA, GSE14520 expression data and sample information are downloaded from the GEO database, and LIRI-JP expression and sample clinical information are downloaded from ICGC database, to screen out 1122 DDR and ICD related genes.

In this example, the Cox regression analysis is as follows:

The single-factor cox analysis: regression modeling is respectively performed on a single gene or a clinical characteristic by using a coxph function of a survival package to screen prognosis-related genes or clinical characteristics based on p<0.01, corresponding modeling parameters are extracted and then a forest map is plotted by using a forest plot package. The multi-factor cox analysis is as follows: regression modeling is performed on the constructed multi-gene or clinical characteristics by using a coxph function of a survival package.

Survival analysis in this example is as follows:

Survival analysis-related genes or clinical pathological characteristic factors are screened, numerical conversion is performed, grouping is performed by using a surv_cutpoint automatic selection threshold of a survminer package, and survival information and grouping information were fitted by using a survfit function of a survival package.

The LASSO regression in this example is as follows:

LASSO regression modeling is performed on prognosis-related genes by using a glmnet function of R package glmnet, and cross-validation is performed on a cv.glmnet function, LASSO screening is performed by using lambda. min as an optimal lambda parameter to obtain 21 genes, wherein a multivariable cox model is screened further stepwise, and 11 genes are finally retained, the multi-factor cox model is constructed using these genes, and a corresponding risk score is calculated.

The independence validation and nomogram of the risk score in this example are as follows:

To validate that the risk score has independent prognostic efficacy, a single-factor cox analysis is performed on the TCGA-LIHC dataset in combination with clinical pathological characteristics: stage, gender, vascular, age, and AFP. Then, the overall prognosis of the above 6 factors (containing risk score) is then analyzed by utilizing multi-factor cox regression to validate the independent prognosis effect of risk score. A cox proportional risk regression model is constructed by using a cph function of R package rms, then survival probability is calculated by using the survival package, a nomogram is finally constructed by using the nomogram function, and a calibration curve is plotted to assess the nomogram and predict accuracy.

For those skilled in the art, the present disclosure is not limited to the details of the preceding exemplary examples and can be implemented in other specific forms while remaining true to the spirit and basic features of the present disclosure. As a result, the embodiments should be regarded as exemplary and non-restrictive from any perspective. The appended claims, not the above description, define the scope of the present disclosure. Therefore, alla changes within the meaning and scope of the equivalent elements of the claims in the present disclosure are intended to be included.

It should also be noted that, while the specification is described in terms of embodiments, not every embodiment only contains an independent technical solution. This specification description provided solely for clarity. Those skilled in the art should consider the specification as a whole, and the technical solutions in each embodiment can also be properly combined to form other embodiments that those skilled in the art can understand.

What is claimed is:

1. A construction method for a prognostic model of hepatocellular carcinoma based on DNA damage repair (DDR) and immunogenic cell death (ICD) gene expression, comprising the following steps:
step 1, acquiring transcription profile expression data of multiple hepatocellular carcinoma patients;
step 2, screening candidate genes based on the transcription profile expression data of the multiple hepatocellular carcinoma patients;
step 3, determining prognostic genes related to lifetime through single-factor Cox regression analysis based on the candidate genes;
step 4, screening the genes related to the lifetime through LASSO Cox regression analysis to determine genes for constructing a risk score model and the risk score model; and
step 5, assessing the prediction performance of the risk score model based on the above training dataset.

2. The construction method of the prognostic model of hepatocellular carcinoma based on DDR and ICD gene expression according to claim 1, wherein the genes for constructing the risk score model comprise: FFAR3, DDX1, POLR3G, FANCL, ADA, PIK3R1, DHX58, TPT1, MGMT, SLAMF6, and EIF2AK4.

3. The construction method of the prognostic model of hepatocellular carcinoma based on DDR and ICD gene expression according to claim 1, wherein step 5 comprises:
calculating a risk score of each subject in the training dataset based on the risk score model;
analyzing the score by using a time-dependent subject working characteristic curve of the training dataset; and
analyzing and evaluating the fitting goodness of the score model by using the time- dependent subject working characteristic curve of the training dataset.

4. The construction method of the prognostic model of hepatocellular carcinoma based on DDR and ICD gene expression according to claim 3, wherein a grouping cut-off value is analyzed and determined according to the time-dependent subject working characteristic curve of the training dataset, and the subjects in the training dataset are divided into a first high-risk group and a first low-risk group according to the grouping cut off value;
whether the first high-risk group and the first low-risk group have a significant difference in survival is assessed by using a Kaplan-Meier curve of the training dataset.

5. The construction method of the prognostic model of hepatocellular carcinoma based on DDR and ICD gene expression according to claim 1, wherein the Cox regression analysis comprises single-factor cox analysis and multi-factor cox analysis.

6. The construction method of the prognostic model of hepatocellular carcinoma based on DDR and ICD gene expression according to claim 5, wherein the single-factor cox analysis is as follows:
regression modeling is respectively performed on a single gene or a clinical characteristic by using a coxph function of a survival package to screen prognosis-related genes or clinical characteristics based on p<0.01, corresponding modeling parameters are extracted and then a forest map is plotted by using a forest plot package;
the multi-factor cox analysis is as follows: regression modeling is performed on the constructed multi-gene or clinical characteristics by using a coxph function of a survival package.

7. The construction method of the prognostic model of hepatocellular carcinoma based on DDR and ICD gene expression according to claim 1, wherein in the LASSO Cox regression,
LASSO regression modeling is performed on prognosis-related genes by using a glmnet function of R package glmnet, and cross-validation is performed on a cv.glmnet function;
LASSO screening is performed by using lambda.min as an optimal lambda parameter to obtain 21 genes, wherein a multivariable cox model is screened further stepwise, 11 genes are finally retained, the multi-factor cox model is constructed using these genes, and the corresponding risk score is calculated.

8. The construction method of the prognostic model of hepatocellular carcinoma based on DDR and ICD gene expression according to claim 1, wherein the independent validation and nomogram of the risk score is as follows:
first, single-factor cox analysis is performed on the TCGA-LIHC dataset in combination with clinical pathological characteristics: stage, gender, vascular, age, and AFP;
second, multi-factor cox regression is utilized to analyze the above 6 factors including the overall prognosis of risk score to validate the independent prognosis effect of risk score;
a cox proportional risk regression model is constructed by using a cph function of R package rms, then survival probability is calculated by using a survival package, a nomogram is finally constructed by using a nomogram function, and a calibration curve is plotted to assess the nomogram and predict accuracy.

9. A prognostic model of hepatocellular carcinoma obtained by using the construction method of the prognostic model of hepatocellular carcinoma based on DDR and ICD gene expression according to claim 1.

10. An application of the construction method of the prognostic model of hepatocellular carcinoma based on DDR and ICD gene expression according to claim 1 in the treatment and prognosis of hepatocellular carcinoma.
